(19) United States
(12) Patent Application Publication
(10) Pub. No.: US 2005/0215848 A1
(43) Pub. Date: Sep. 29, 2005

(54) Method and apparatus for the temporal synchronization of meditation, prayer and physical movement

(76) Inventor: Raymond M. Lorenzato, Paradise, CA (US)

Correspondence Address: Ray K. Shahani, Esq.;Twin Oaks Office Plaza, Suite 112, 477 Ninth Avenue, San Mateo, CA, 94402-1854, US

(21) Appl. No.: 11/137,529

(22) Filed: May 25, 2005

Related U.S. Application Data
(63) Continuation-in-part of application No. 10/618,981, filed on Jul. 10, 2003.

(51) Int. Cl. A61M 21/00

(52) U.S. Cl. 600/27

(57) ABSTRACT

The present invention is the method and apparatus for providing sound or visual cues to provide the synchronization in time of groups of individuals in meditation, contemplation, prayer and physical movement. The sound or visual temporal cues can be integrated into wristwatches, clocks, communication devices such as phones, networked computer devices including computers, entertainment processes including television and radio broadcasting, and information management tools such as PDAs, or be integrated into an appliance dedicated for the purpose of synchronizing said activities. The user of said devices experiences a sound or visual cue at one or several given times a day. On the cue the patient consciously takes some moments to engage in said activities. As a group of individuals are using the same moment to engage in the same or similar activities the user may feel a sense of belonging to the group. If the user wishes to participate in the activities in synchrony with other individuals he or she may experience a sense of contentment or happiness at this time. In one embodiment, downloadable mantras are available over the internet or through another network, providing a selection of downloadable mantras including audio and visual files, which can be downloaded and installed to a local electronic device, such as a cell phone or PDA.

### RELATED APPLICATIONS

This Application is a Continuation-In-Part of related pending U.S. patent application Ser. No. 10/618,981 filed Jul. 10, 2003 entitled METHOD AND APPARATUS FOR THE TEMPORAL SYNCHRONIZATION OF MEDITATION, PRAYER AND PHYSICAL MOVEMENT, which is incorporated herein by reference in its entirety, and claims any and all benefits to which it is entitled therefrom.

### FIELD OF THE INVENTION

The present invention relates to the synchronicity of meditation, contemplation including prayer and physical movement, yoga, martial arts, dance, exercise and song of individuals in diverse physical surroundings, and more specifically to the use of images, sequences of images, colored signals and sounds designed and orchestrated to facilitate the cuing of said activities in individuals wanting or desiring or needing cues from mechanical and electronic devices such as watches, mobile phones, personal desk accessories, computers and internet devices for this purpose.

### BACKGROUND OF THE INVENTION

The use of group meditation, synchronized mostly by ritual cues and/or verbal instructions, has over centuries been central to the spiritual buoyancy of many cultures. Meditation, contemplation including prayer and physical movement may be engaged as a individual practice or as a group practice. In the practice of meditation sounds and or images may be made which focus the person or persons meditating on the spiritual realm. The unity of a group in meditation may be defined as the process of the group meditating at the same time. Attempts to synchronize said activities vary with cultures and individuals.

Originally synchronization of these activities depended on an identified individual cuing an audience of practitioners to participate. Discussion of prior art is with reference to varied embodiments of the present invention.

Murata addresses the distribution of propitious information in U.S. Publication No. 20020009988. An information serving terminal is operated by a worship place and a wireless terminal for distributing the information. Murata is designed to simulate an actual visit to a place of worship. Murata is used in delivering prayers to individuals when an individual wishes to acquire a prayer. Murata is never described as attempting to create a cueing of meditation, contemplation including prayer or physical movement in order to synchronize these activities in a temporal sense and the information emanates from a place of worship.

Birnbach et al. describes an invention for delivering prerecorded psycho-suggestive messages. In discussing the background of their invention the benefits of “positive thinking” are briefly discussed without acknowledging the psychological implications of reinforcing positive affects on the organization and development of an individuals personality. Bimbach et al may be explained by understanding the nature of positive affects of belonging, security, faith on balancing defensive affects of personality including fear, control and others in order to maintain the defenses relaxed sufficiently to not manifest as anxiety. The invention of Bimbach et al. does not synchronize users in meditation, contemplation including prayer, physical movement including dance, martial arts, yoga and song.

The patent by inventor Gehlot on Jun. 19, 2001 (U.S. Pat. No. 6,249,222) describes an apparatus and method for generating a color based alerting signal to alert individuals to the occurrence of a predetermined event. The example of an incoming telephone call or page is used as a predetermined event. A colored base signal generated in response to a predetermined event such as an incoming call is distinct from a colored base signal that is an integral part of the mechanism of a device designed to cue an individual at one or many given times.

In contradistinction with the prior art, there is a fundamental difference between cueing individuals to initiate carrying out an action and alerting a user to the occurrence of a predetermined event such as a phone call. When a cue is generated internally in a device, in the temporal cueing of individuals in order to synchronize the activities of meditation, contemplation including prayer and physical movement the color based signal, when used, is the determined event rather then the response to a predetermined event. In the event that the cue to said actions is in the form of a phone call or digital messaging then the cueing are considered the predetermined event. Other devices generating alarms of other kinds including sounds and vibration for the purpose of alerting a user to the occurrence of a predetermined event are also distinct inventions from the present invention.

There is no suggestion in the prior art to synchronize meditation, contemplation or physical movement of a group of individuals in diverse physical surroundings. While from time to time, television and radio has functioned to identify an individual to cue an audience of practitioners to participate in the aforementioned processes and activities, the present invention uses sound and visual images to synchronize said activities. These sounds and visual images may also be broadcast by television and by radio.

Dahl describes in U.S. Pat. No. 6,326,881, an alarm clock system. The embodiment of the present invention is not merely a clock, but rather, is dedicated to providing various cues to meditation and the aforementioned activities of the present invention at a prescheduled times and scheduled times set by the user. The invention by Dahl at random times notifies an individual user of the device when it is time to become conscious of a moment facilitating a mindfulness, an awareness and stress reduction and quiet at that time. The present invention facilitates mindfulness, greater awareness, stress reduction and a tendency to be quiet in some and to sing in others; yet the means achieving this are quite distinct, whereas Dahl makes no attempt to create a unity of these activities in a group of persons who are physically in distinct surroundings.

### ADVANTAGES AND SUMMARY OF THE INVENTION

Thus, it is an object and advantage of the present invention to provide a method and apparatus for the temporal synchronization of meditation, prayer and physical movement.

Another object and advantage of the present invention is to facilitate the temporal synchronization of the process of meditation, contemplation including prayer and physical movement including dance, yoga and martial arts for those individuals interested in synchronizing these activities with other individuals. The invention uses sound and visual cues from a myriad of technical devices synchronized to broadcast at one or multiple times a day in order to achieve the synchrony of activities in a group of individuals in distinct physical surroundings.

Other objects, advantages and features of the present invention will become apparent from the following detailed description considered in conjunction with drawings used to conceptually illustrate the method and apparatus of the present invention.

Further details, objects and advantages of the present invention will become apparent through the following descriptions, and will be included and incorporated herein.

The present invention proposes a novel utilization of a myriad of preexisting technology to achieve a means to create synchrony of said activities. No prior invention serves the explicit purpose of creating a temporal unity of meditation and/or contemplation including prayer and/or physical movement including yoga, martial arts, dance and exercise over physical distance.

In a preferred clock or wristwatch embodiment, the use of acoustic transducers which are water and pressure resistant, the use of liquid crystal displays and of digitalized sound stored in ROM or EPROM are more recent developments in watch development that will be utilized in design and production.

Embodiments of the present invention designed to synchronize individuals in said activities include but are not limited to the following:

1) A watch or clock with an information system internal to the watch which includes a logic circuit, memory storage system and a means to deliver sound and or visual cues to the wearer of the watch at specific times. Said device may contain, either singly or in combination, a speaker for broadcasting sounds, and a screen for viewing images. The device shall have one or several input devices for managing the information and functions of the device.

2) A watch or clock with the capacity to receive telecommunication signals with part of the information system peripheral to the watch and supplied through telecommunications.

3) Software and firmware in telecommunications devices used to produce sound cues replacing phone alarms that may be activated by “phone calls” designed not to be responded to other then by beginning meditation or other of the aforementioned activities at that time. These “phone calls” can be conducted en masse resulting in a large number of individuals receiving the cue at the same time.

4) Software and firmware in PDAs, personal computers and internet devices manifest as graphic frames that pop ups on a devices screen without any immediate prompting by the user. Said “pop up” graphic images may be distributed software that resides in the user's computer or software that is distributed by a server computer in a network of computers. The “pop up”s may or may not be accompanied by sound cues.

5) A dedicated device which is designed to prompt or cue meditation a specific times during the day. An example of this is a device that chants “Peace” every 6 hours for a given duration of time. The device would allow the user to add or delete meditation cueing times, alter durations, and change the tones, language or words chanted. This facilitates large portions of a population to meditate or contemplate in unity at those same times.

Many watches, clocks and other devices have integrated into their function alarm systems. In the present invention, devices will be constructed specifically for the purpose of alarming an individual to an event. The process of synchronizing individuals in said activities over physical distance could be achieved by means other then the present invention, nonetheless the present invention is useful, novel and will produce new and unexpected results. Alarms used, unlike those in the prior art, will not have agitating or irksome tones so that the likelihood of induced meditation and contemplation are enhanced.

In an another embodiment of the present invention, phone calls used to cue meditation and contemplation at specific times of the day, eventually even other agitating sound tones that otherwise would result in the physiological response of vigilance and alertness will result in relaxation and finding the capacity to meditate within. When specific sound tones in a telecommunication or internet or other type of linked device are related specifically to said cueing of meditation process, including telephone calls, “You've got mail” and other, mono- or poly-phonic tone common or ubiquitous or unique sounds, images and sensations, then a distinct physiologically healing or meditation-inducing response to those sound tones, images or other sensations would be expected as the individual becomes conditioned to those tones, images or other sensations.

Use of the present invention will be facilitated by distribution of information on the benefits of unity in meditation.

In the case of the preferred embodiment of the present invention, the psychological relationship or empathy with the meditation watch establishes the novel attributes of being a focal point for cueing synchronization of meditation and/or contemplation including prayer and/or physical movement. Cues for meditation and the other aforementioned activities contain emotional content and are therefore “psycho-suggestive messages”.

Cues of aforementioned activities referred to in the present invention are designed to initiate a myriad of activities; contemplation and meditation are included. The content of a cue is short and not specific. In the present invention it is up to the user to determine the message of his or her activity. For example, by creating a synchrony of meditation a unity in meditation is established allowing the user to understand he or she belongs to a greater whole. In the present invention, faith is directed outwardly by knowing that other individuals are engaged in psychological, spiritual and social growth rather then inwardly to generate new self images. The present invention provides a solution for individuals wanting to meditate in unity. Users are seeking a reflection of their own moods and state of mind at time of activities rather then a cognitive modification of thought processes. They are relating to the deeper affects of emotional states rather then processes of rationalization such as “personal circumstances” and “personal challenges”.

Embodiments of the present invention include enhanced alarms functions on watches, mobile phones, personal desk accessories, internet devices and computers. Alarms that are specifically designed for the function of synchronizing meditation, contemplation and physical movement of a group of individuals that can synchronize these activities without additional instructions and communications in the uses of these alarms. Images that exist on, and sounds that emanate from watches, mobile phones, personal desk accessories, internet devices and computers are designed specifically to synchronize meditation, contemplation and physical movement fail to create an identity with individuals of the device with the purpose of synchronous meditation, contemplation or physical movement. The use of devices for a given purpose is dependent on individuals recognition of that device for that given purpose. When individuals do not feel that the purpose of an alarm on a watch is to synchronize meditation and the rest, then it will not be used for such. The probability exists that meditation, contemplation and physical movement occur more often on the hour due to tendencies of individuals to set alarms on the hour, yet it is not the intension that the use of the alarm mechanism to create synchrony of these events and the expectation of individuals wanting to synchronize these events can not depend on this chance. In the users, understanding that their efforts to synchronize meditation, contemplation and physical movement will be mirrored by the efforts of others individuals will facilitate synchronizing these activities.

In a preferred embodiment, the device comes to the user pre-programmed with cues set at 6:00 am, 12:00 noon and 6:00 pm. The user is encouraged to use these times as well as other set times in order to effect a unity of meditation and the aforementioned activities at these times.

The present invention solves the problem of providing a group of individuals, who wish to be temporally synchronized in meditation, contemplation including prayer or physical movement, a process facilitating their wants irrespective of their physical distance from each other. The present invention also provides a simple means to synchronize said activities to those individuals who had not prior considered the possibility.

The present invention may result in an increase in the compliance of individuals wanting to practice the aforementioned activities on a regular basis. Benefits of said activities in synchrony with other individuals may partially be due to an increased amount of time spent in said activities.

The psychological benefits of belonging to a group of individuals focused on positive sentiment may be demonstrable. Western psychoanalytic theory suggests that personality is defined largely by the conscious subjective aspects of emotions as they are influenced by genetic and environmental influences. The manifestation of personality is frequently considered the accumulative modification of innate effects in defense against pain and suffering. The accumulative modification of innate affects in constructive patterning may be understood in the reinforcement of faith, the sense of belonging to another individual and/or a group and/or a higher power and the sense of security maintained by the individual and group. Synchronization of meditation, contemplation including prayer and physical may promote constructive as opposed to defensive affects. Consequently the benefits of said activities in synchrony with other individuals may also be due to an accumulative modification of innate affects in constructive patterning by the practice of faith, a sense of belonging and a sense of security resulting in the promotion of positive affects.

Personality disorders such as narcissism, and malignant narcissism, and borderline personality disorder may have beneficial therapeutic outcomes when individuals dominated by these disorders engage in the practice of reinforcing the positive affects (faith, the sense of belonging and sense of security) and when they practice compassion. It is understood that the outcome of anger and rage that preoccupies the brain for an instance may result in a modified outcome when individuals learn to integrate these affects with cortical reasoning. The capacity of individuals to modify their reflexes of acting out anger and rage may be modified by meditation and contemplation. The tendency for individuals with a predisposition to narcissism to use material goods as narcissistic extensions of themselves may be modified by reinforcing constructive affects resulting in more responsible materialism. The present invention promotes the positive sentiment of faith, a sense of belonging to a group of individuals involved in a unity of meditation and in this way may be therapeutic in individuals with narcissistic and borderline tendencies. It provides a harmonious resolution in individuals wanting to be cued in mediation one or more times a day.

Individuals with anxiety disorders and manifestation of anxiety such as panic disorder may benefit from the periodic relaxation that may result from engaging in the aforementioned activities. Cognitive modification resulting in healing from anxious states may result if an individual identifies a time to relax and maintains moments of full awareness on a periodic basis. Individuals who suffer from obsessing with their attachment to persons and worldly goods may benefit if they use moments of contemplation to acknowledge the transient nature of all things.

When a person says that he or she has been in meditation, contemplation including prayer of physical movement and wants to be in a shared process of this same activity we can understand this to be truthful for the individual. This invention may accommodate that truth.

The benefits of synchronizing dance, exercise and physical movement of a group of individuals may be greater then the sum of the physical benefits to each individual. This would be understood in the sense of belonging to the group that may be created and the psychological benefits of this.

Other health benefits including strengthened immune systems have been suggested to exist in individuals who meditate regularly.

Benefits of said activities in synchrony may result in individuals developing a capacity to have faith that other individuals are involved in said activities. By exercising faith and belonging, which are positive affects of personality, personality structure of the culture may evolve away from malignant narcissism where the material world is embraced without a sense of responsibility to a realm of responsible materialism compatible with the practice of compassion for present and future generations.

If the present invention is produced with times of cuing set at 6:00, 12:00 and 18:00 and individuals continue to carry out their activities and processes at these time, then all the individuals, who where awake, in the given time zone and who used cuing devices would be synchronized. Assuming 24 time zones in the world, three other time zones would also be synchronized with any given time zone.

The present invention is the method and apparatus, using components of electronic and/or mechanical devices contained in part within or completely within watches, personal desk accessories, portable phones, computers, networked devices or appliances, to temporally synchronize meditation and/or contemplation including prayer and/or physical movement including dance, martial arts, yoga and song in individuals, in shared or diverse physical surroundings.

The benefits of the invention are many including providing those individuals who want to be synchronized in said activities the opportunity for this. The individual and social benefits of the invention may include the therapeutic modification of narcissistic and borderline personality disorders in individuals. This may result in more responsible material consumption in some individuals as well as a more consolidated sense of self in these individuals.

The preferred embodiment of the present invention is in the form of a watch which cue users in the aforementioned activities by broadcasting a sound and displaying the graphics on an LCD several predetermined times a day. Cues to the aforementioned activities are set at 6:00 am, noon and 6:00 pm. The user may add and/or delete cues.

By using mechanical and/or electrical means on devices such as watches, mobile phones and internet devices, computers, timed sounds and or images emanate from, or appear on, the devices at one or more times in a day. Synchronization may be to particular times in the day or particular events in nature such as the time of sunrise at a given location. When meditations are synchronized to each hour of a 24 hour clock then awake individuals throughout the world would find unity of meditation with other individuals users on the hour. If users maintain the preset times of cues then users will be synchronized with other users in the same time zone and with users in time zones 6, 12 and 18 hours advanced or delayed. If users wish to be synchronized with users in the continental United States it is suggested that they synchronize with 6:00, 12:00 and 18:00 Pacific Coast Time. Users not within the Eastern Standard Time zone may set their cues both in order to be cued with other users in their time zone, and with users within the Eastern Standard Time zone.

It is a further object and advantage of the present invention to provide a downloadable mantra or meditation minder. Such downloadable mantra consists of downloadable audio and visual files, i.e., any of various types of graphics and audio files.

It is an object and advantage of the present invention to provide a website or other electronic database which consists of an interface in which users may select a preferred mantra, i.e., combination of sound and graphics files.

It is a further object and advantage of the present invention to provide downloadable mantras which may be installed and operated on local electronic devices, including cell phones, PDAs, etc. installation and operation may also include setting or adjusting the audio features, any graphics or video selections, setting ring times and durations, frequencies, etc.

Numerous other advantages and features of the present invention will become readily apparent from the following detailed description of the invention and the embodiments thereof, from the claims and from the accompanying drawings.

### BRIEF DESCRIPTION OF THE DRAWINGS

The invention is illustrated below and represented schematically in the following drawings:

FIG. 1 is a representative functional block diagram of a preferred embodiment 100 of the present invention.

FIG. 2 is a drawing of a preferred embodiment of the present invention comprising a watch with the additional function of cuing the user with sound or visual cues to one of the aforementioned processes or actions.

FIG. 3 is a drawing of another preferred embodiment of the invention in which a dedicated device has no other function then to provide temporal cues to the aforementioned processes or actions.

FIG. 4 is a schematic of a preferred embodiment of the invention that receives broadband signals or signals from a network of computers or appliances in the process of cuing the user to the aforementioned processes or actions.

FIG. 5 is a schematic of an embodiment of the invention that receives broadband signals or signals from a network of computers or appliances in the process of cuing the user to the aforementioned processes or actions, in which control to regulate cues is effected at the levels of the cuing device and through a database which in part determines the incoming signals to the cuing device.

FIG. 6 is a schematic of the menu options used by an individual to input information into the cuing device wherein the menus may appear on the screen on the device itself; as in the case of the preferred embodiment: a wristwatch with the input mechanism comprising two buttons, the crown and a screen, or the menu items my be used to make selections on a computer or other device and the resulting selections downloaded to the cuing device.

FIG. 7 is a schematic of the populations of users of the present invention 100.

FIG. 8 is a representative diagram illustrating an embodiment of a screenshot of the homepage 800 of a prototypical website of the present invention, such as the NNNOOOWWW project.

FIG. 9 is a representative diagram illustrating a preferred embodiment of a screenshot of a web page or user interface for a synchronization apparatus adapted for use with a cellular or other mobile phone 900.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

The term “device” used within this patent application does not suggest that the elements contrived and designed as parts of this invention need be adjacent or in proximity to each other, only that they are interrelated.

### List of Reference Numbers

11: Timer Module
12: Central Processing Unit and Memory Module
13: Logic Circuitry
14: Speaker or sound generating device
15: Screen or visual display unit
16: Graphic generation circuit
17: Sound generation circuit
18. Input mechanism
21. Arm of the user's of a cuing device watch
22. Cuing device watch
23. Crown
24. Input mechanism button
25. Input mechanism button
26. Internal speaker
27. Screen
28. Representation of sound
31. Housing
32. Speaker
33. Screen
34. Input mechanism knob
35. Input mechanism knob
36. Electrical cord
37. Electrical cord plug
41. User of cuing device
42. Regulating mechanism
43. Cuing device
44. Other user devices
45. Sound cues
46. Graphic cues
47. Device timing the broadcasting of signals
48. Incoming signals
49. Device producing the broadcasting of signals
51. Database of users and scheduling of cues
61. Menu showing list of cues
62. Menu initiating the establishment of a cue
63. Menu utilized in deleting a cue
64-68. Menus used to set the time of a cue in establishing or deleting a cue
69. Menu used to select a choice of graphic images or animations used in cuing
70. Menu used to select the number of times the selection of Menu 69 will be repeated on each cue
71. Menu used to select a choice of sounds used in cuing
72. Menu used to select the number of times the selection of Menu 71 will be repeated on each cue
81. Users cued in activity or process by devices with internalized timing, scheduling and presentation information for cues.
82. Users cued in activity or process by devices receiving distributed information over broadband for timing and/or scheduling and/or presentation of information for cues.
83. Users cued in activity or process by devices receiving distributed information over a network or networks for timing and/or scheduling and/or presentation information for cues.

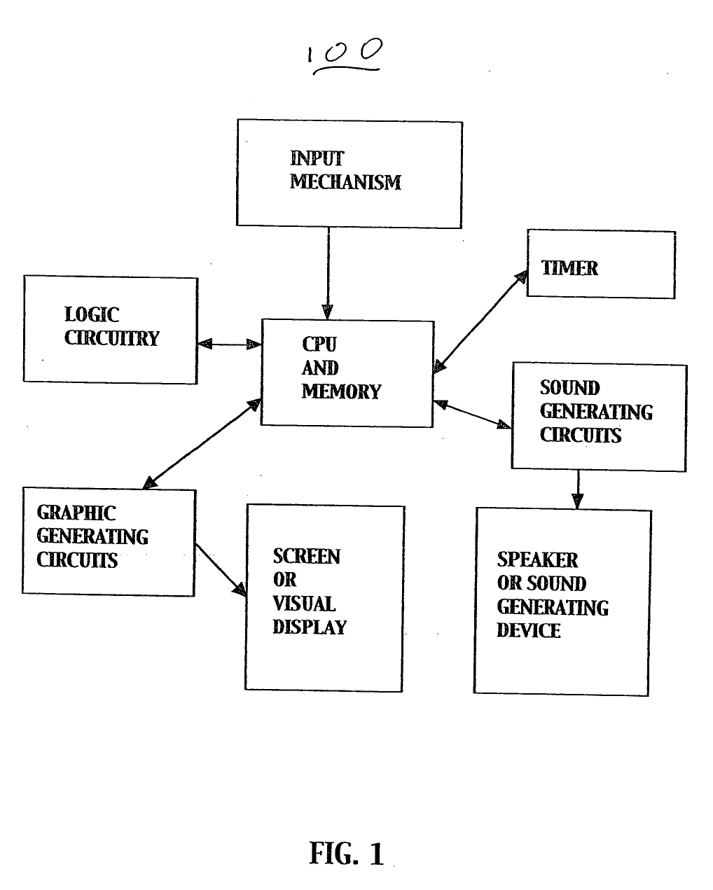

FIG. 1: The user of the present invention is cued to the aforementioned activities by graphics on 15 the screen or a visual display such as an image lighting up or by sound from 14 a speaker or sound generating device. The input mechanism 18 may include mechanical devices such as buttons on the side of a watch 24 and 25, or dials on a housing 34 and 35, or keyboards of computers and electric appliances such as mobile phones and PDAs, and other mechanical devices, and may be menu driven by computer programs that use sound or other input devices. The input mechanism 18 may provide the 12 CPU and Memory, with information that is organized in a way minimizing the use of CPU and or minimizing the use of memory in the device, or that utilizes the CPU and Memory to the fullest.

The invention takes various forms. Devices that incorporate the invention include, but are not limited to watches, mobile phones, PDAs, personal computers, networks of computers, radios and televisions. The distribution of the components of the invention 18, 11, 12, 13, 16, 17, may be within the user device, as in a watch or distributed between devices, as in networks of computers and radio broadcasts. The presence of all components in FIG. 1 are not essential to the invention. Graphic or sound cues are produced by the invention with 16 the graphic generating circuits and 15 the screen or visual display and 17 the sound generating circuits and 14 the speaker or sound generating device respectively. A visual display may exist without the use of a screen, 15. When the whole or part of a face of a watch illuminates, showing the design of the watch, this could be considered a visual display for cueing the aforementioned activities. The most common embodiment of the present invention contains a speaker as part of the 17 sound generation circuit, and a screen, 15 as part of the visual display.

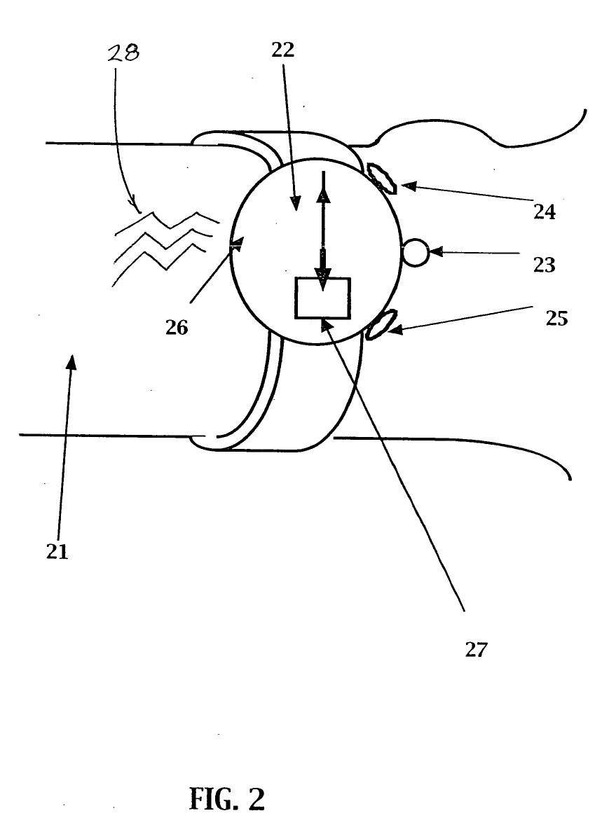

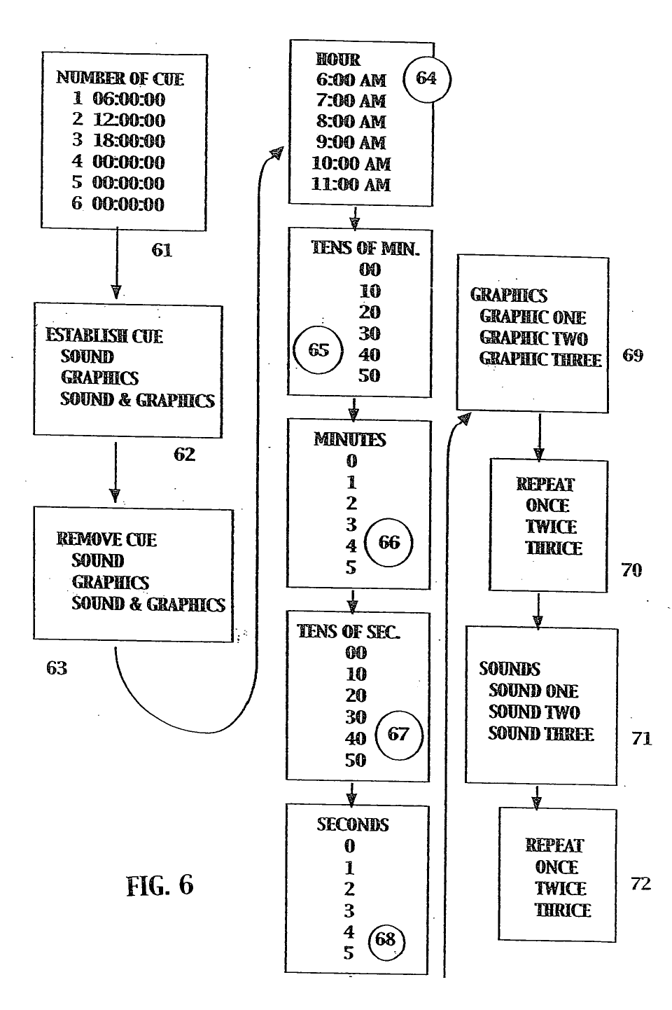

In the preferred embodiment of the present invention the input mechanism 18 consists of menu items 61-72 and input mechanism buttons 24,25 (See FIG. 2 and FIG. 6).The combination of the logic circuitry 13, and the CPU and memory, 12 allows the user to select and activate (store for use by the device) the information. The timer module 11 may be internal to the device (a watch or computer) or external (radio or television). The presence of a timer does not suggest that the invention need function as a clock or watch. A mobile phone may use and internal or external timer. A computer, mobile phone, networked PDA, or other device on a network may use an internal or external timer 21.

The CPU and memory module, 12 stores the information used to construct sound and images, a list of selections of the choices of the user and the times and durations of the cues. The CPU and memory module 12 may be internal to the device or in part distributed over a network. The logic circuitry 13 determines the course of information that is input into the device and the outcome of that information. It may be constructed of both firmware and/or software.

FIG. 2 is a drawing of the preferred embodiment of the present invention. The preferred embodiment is a watch, 26 which has the additional function of producing cues to the aforementioned activities. The watch is initially set to produce a cues at 6:00, 12:00, and 18:00 hours. The watch is initially set to produce two repeats of the sound and two repeats of the graphics with several seconds between each sound and graphic. The sounds and graphics occur at approximately the same time. Input mechanism buttons, 24 and 25 allows the user of the watch to modify the times and types of cues to the aforementioned activities. The watch, 26 has a crown, 23 which allows the user to set the time of day. To set the time of day the crown is pulled outwardly from the body of the watch. When the crown is pulled out the second hand is disengaged. The minute and hour hands may be reset by twisting the crown. To engage the hands in time keeping activity the crown 23 is pushed inwardly. The times and type of cues to the aforementioned activities are set by use of buttons, 24 and 25 and the screen, 27. The choices of sound and graphic cues are made through interacting with a scrolling menu 61-72 (FIG. 6) on the screen. When a given time of cue occurs the device produces a sound 28 and/or graphic cues on the screen 27 for meditation or other aforementioned activity. The menu selection establishing or removing a sound and/or graphic cue may be repeated several times until the schedule of cues is established for the user of the device.

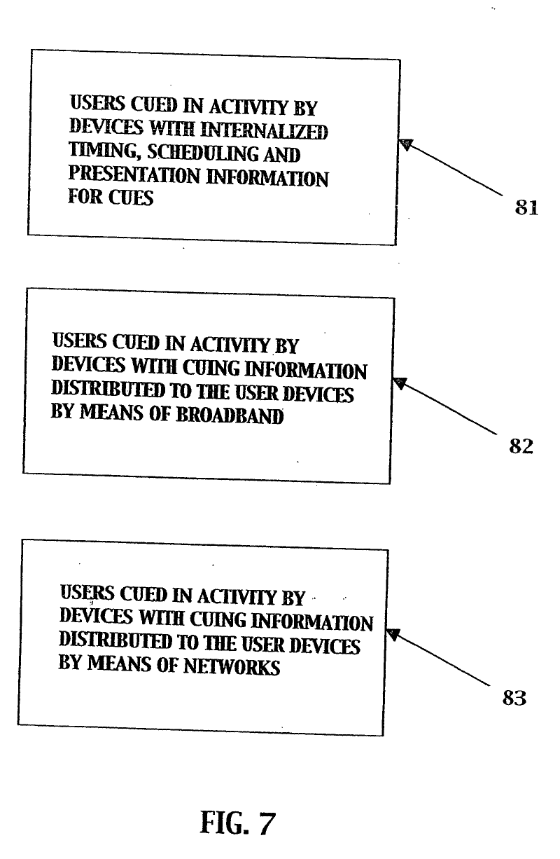

When both of the input buttons, 24 and 25 are pushed at the same time the menu selections toggle appear and disappear on the screen, 27. FIG. 7 is a schematic of the menu selections showing on the screen.

Pushing either button 24 or 25 by themselves has distinct results if the menu is showing or is not showing. When the menus is not showing and a sound cue is in the process of being broadcast, pushing either button 24 or 25 will immediately silence the broadcast of the cue. Any repeat of the cue that might follow within the present sequence of cuing will be silenced as well. This action does not delete the cue from the menu. The next cue beyond the present sequence will continue as normal cues, with repeat sounds or images as scheduled, unless the user enters the menu and deletes or adds cues.

When the menu is showing pushing button 24 results in the menu scrolling down one item at a time. Pushing button 25 by itself results in activating the window item selected.

When the menu is scrolled to its completion by button 24 and without pushing button 25 during the entire scrolling of the menu, then the menu will disappear. Scrolling the menu and activating menu items may result in following the menu through some branches rather then linearly. In the event that an item is selected in box 62 (sound or graphics or sound & graphics), then the screen will scroll immediately to the time (64-69) to facilitate setting the time and will bypass the “Remove Cue” box. Subsequent to setting a time the menu will continue box 69 to 72 to complete the menu. Upon completion of menu item 72 the screen clears of the menu. Holding down buttons 24 and 25 will cause the menu to return. Each time a cue is added or deleted menu 61 updates the list of cues. The time listed on menu 61 refers to the beginning of each cue.

Users are encouraged to maintain at least one of the preset times of cue activation in order to synchronize their activities with a large population. Users may be encouraged to set one or more of their cues to specific times such as 6:00, 12:00 and 18:00 Pacific Standard Time (9:00, 15:00 and 21:00 Eastern Standard Time).

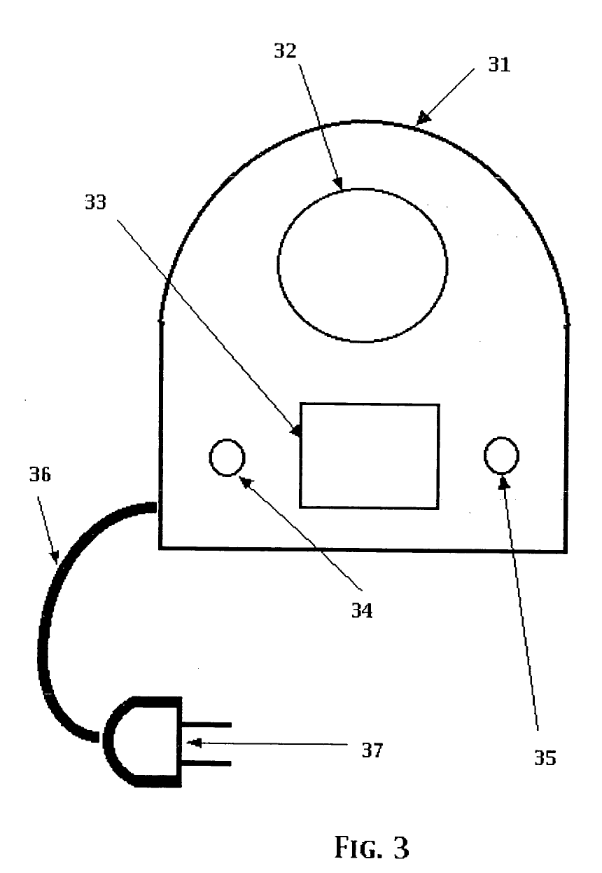

FIG. 3 is a drawing of an embodiment of the invention. It is a dedicated device. The device has no other function then to provides temporal cues. The purpose of the temporal cues are to facilitate a unity in meditation and the other aforementioned activities. The embodiment in FIG. 3 contains the components: the housing, 31, the knobs (input devices 27 and 28), a screen 25, a speaker 24 and a cord to a power source. The device contains most of the components of a clock, yet no display of the time is evident. The form of the device must provoke an empathetic response compatible with its use. In the event of the use of the dedicated device is to cue meditation, a form of the housing 31 compatible with meditation is desirable. A housing designed with values of warmth and familiarity or suggesting an altar may best suit the mood and attributes of a user engaging in meditation. In the event of the use of the dedicated device is to cue dance or song, themes of dance and song may illustrated the housing 31. The form of the housing may be a sculpture of a figure engaged in dance for instance; in the case of the dedicated device being used to cue dance. The power source of alternating current and the use of a cord to connect the power source to the device may be substituted by batteries or power cells. The user uses input device 27 and 28 to scroll and select menu items on the screen. The speaker broadcasts sound cues and the screen displays graphics at the time of cues for the aforementioned activities as selected by the user. The device may come with cues for the aforementioned activities set at the hours of 6:00, 12:00, and 18:00 with the suggestion that the user maintain one or more of these time in order to facilitate the temporal synchrony of cues with a large number of individuals.

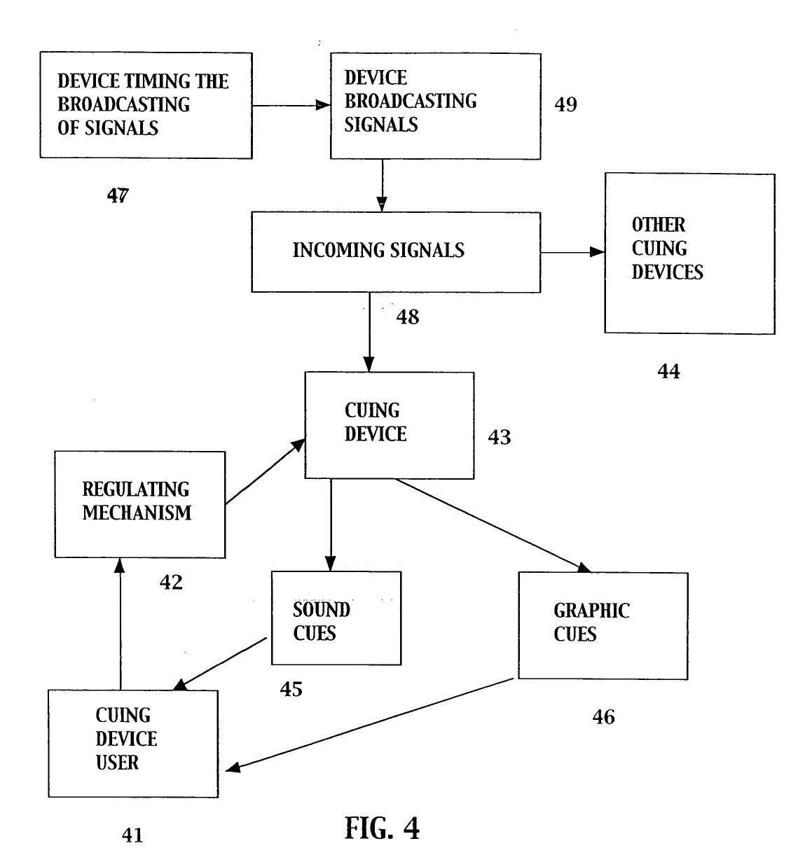

FIG. 4 is a schematic of an alternative embodiment of the invention. The embodiment of the invention in FIG. 4 receives broadband signals. The broadband signals may contain information specifying the timing and form of the cues. The broadband signals may be radio frequency waves containing the information about sound cues or television or mobile device frequency waves containing information about graphic and/or sound information. The present invention, as noted in FIG. 1, contains input mechanism 18, CPU and memory 12, Timer 11, logic circuitry, sound and graphic generating circuits 17 and 16. The flow of information over these elements may be distributed over broadband or networks and supply information to the cuing device 43 or they may be internal to the cuing device. FIG. 4 illustrates a device timing 47 the broadcasting of signals 47, and the production 49 of incoming signals 48 to the one user's cuing device. The devices timing 47 the broadcasting of signals 48, the production of signals 49 also broadcasts the same incoming signals 48 to other cuing devices. The device timing the broadcasting of signals and the device producing signals may be distributed or as one unit. A cuing device may be used by an individual or more one individual and may be networked with other cuing devices. The regulating mechanism 42 allows the user to select sound cues, graphic cue and scheduling and duration of cues at the level of the cuing device.

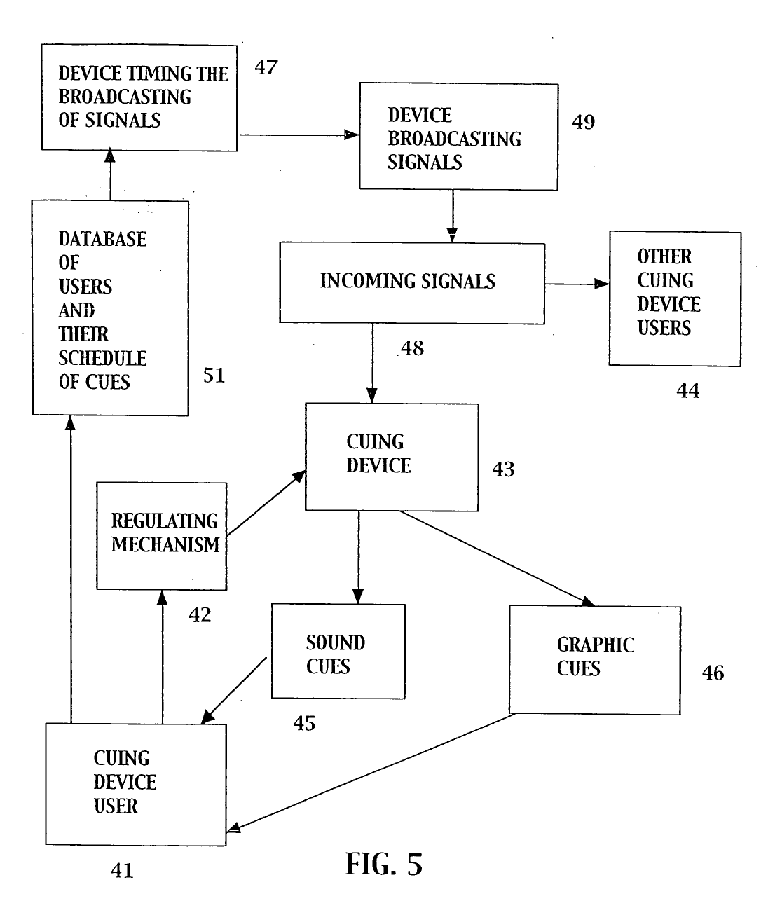

FIG. 5 is a schematic of an alternative embodiment of the invention. The embodiment of the invention in FIG. 5 include elements which time and schedule the broadcasting of broadband signals. A database of user's and their schedule of cues may include timing and content of broadband signals specifying the timing and form of the cues. The broadband signals may be radio frequency waves or television or mobile device frequency waves containing information about graphic and/or sound information. The present invention, as noted in FIG. 1, contains input mechanism 18, CPU and memory 12, Timer 11, logic circuitry, sound and graphic generating circuits 17 and 16. The flow of information over these elements may be distributed over broadband or networks and supply information to the cuing device 43 or they may be internal to the cuing device. FIG. 4 illustrates a device timing 47 the broadcasting of signals 47, and the production 49 of incoming signals 48 to the one user's cuing device. The devices timing 47 the broadcasting of signals 48, the production of signals 49 also broadcasts the same incoming signals 48 to other cuing devices. The device timing the broadcasting of signals and the device producing signals may be distributed or as one unit. A cuing device may be used by an individual or more one individual and may be networked with other cuing devices. The regulating mechanism 42 allows the user to select sound cues, graphic cue and scheduling and duration of cues at the level of the cuing device.

FIG. 6 is a schematic of the menu options used by an individual to input information into the cuing device. The menus may appear on the screen of the device itself, as in the case of the preferred embodiment; a wristwatch with the input mechanism comprising two buttons, the crown and a screen, or the menu items my be used to make selections on a computer or other device and the resulting information downloaded to the cuing device. In the case of the preferred embodiment of a watch with two buttons, a crown and a screen; one button is used to scroll the menu items and the other button is used to activate the menu item.

Item 61 of FIG. 6 represents a list of multiple cues that are active and inactive on a cuing device. Six cuing times are illustrated in item 61. The invention is not limited to 6 cuing times and a list of cuing times is not an essential part of the invention, nonetheless item 61 illustrates a convenient manner of maintaining an understanding by the user of the cuing times used by the user. It also facilitates an understanding by the user of the cuing times that are preset at the factory. Menu 61 may be a scrolling menu containing greater then 6 entries. A menu containing 24 entries; one for each hour, is an example of the schematic representation of item 61.

Item 62 of FIG. 6 represents a menu used to add a cue to the list of cues. The menu choices listed are not a prerequisite of the cuing device. An embodiment of the invention might consist of a cuing device with only sound or only graphic cues. In the event of either of these embodiment the schematic represents a decision to add a cue, not the additional decision of what class of cue to be added. After a menu item is selected, it may be activated by use of some input device. Design process may result in the above choice being activated from one or several menus albeit they are schematically represented as the one menu.

Item 63 of FIG. 6 represents a menu used to delete a cue. In embodiments of the invention with one class of cue the selection is limited to a deleting function only. Embodiments with choices of classes of cues, the choice to delete and the class to delete are both represented. Design process may result in the above choice being activated from one or several menus albeit they are schematically represented as the one menu.

Modules 64 through 68 represent menus used to select time. Other schemes used to select time should be considered to be represented by the sequence of menus modules 64 through 68.

Module 69 and module 71 facilitate the selection of a class of cue. A second tier of class of cues including broadcast or stored graphics and sounds may be used. Classes of graphics may include but are not limited to still graphics including mantras, animations, photographs, image streams. Classes of sound may include but are not limited sounds including mantras, chants, songs, prayers, sounds of nature, music and words and parts of above. Humor may be suggested by the cue. Laughter may be promoted by the cue.

Modules 70 and 71 represent the ability of the user to determine the number of times the user selected for one or two repeats in order to better orient the user in a spacing of silence between sounds. Some embodiments of the invention may facilitate a variety of sounds or graphics in progression for each cuing session.

FIG. 7 is a schematic of a population that is cued in contemplation or other of the aforementioned activities by various embodiments of the present invention. The users of the devices may create a Unity In Meditation (TM); a unified body of individuals in the process of meditating at the same time as a means of defining a state of being. A population of individuals cued to the same or similar activities may have a sense of belonging to a larger group cued to a variety of activities. The intension of the invention is to facilitate the propagation of non violent, healthy sentiment. This may manifest in many ways including song, dance, prayer, meditation and other ways. Populations may be unified in the manifestation of positive sentiment as well as in their specific activities.

Module 81 represents the body of users of devices with internal information management of the cuing schedule and content. An adjunct to a device with internal information management may be a computer program and computer including peripherals. Users of watches with the input mechanism internal to the watch or partially residing in a computer are examples of populations of users represented by module 81.

Module 82 represents users of devices with cuing information distributed to the user devices by means of broadband. Users watching television and being cued in meditation when sound and graphic cues for meditation are broadcast in order to create a Unity In Meditation (TM) is an example of a population defined by module 82. Users receiving cues on mobile phones with or without unique sounds cues are an example of users represented by module 82. When mobile phone users receive a sound message including a “ring” at specific times in a 24 hour period this may constitute as a cuing of meditation.

Module 83 represents users of devices with cuing information distributed over networks. Computer users on the internet receiving cues for the aforementioned activities at specific times represent an example of a population of users defined by module 83.

Downloadable Meditation Minder Application:

The following is a description of an embodiment of a downloadable meditation or prayer graphic and audio files of the present invention. By way of example only, and not intended to be limiting in any way, the following is related to a typical website which will provide the products and services described herein.

Introduction:

The purpose of the NNNOOOWWW project is to synchronize in time the acts of meditation, contemplation, prayer and physical movement in those individuals around the globe wishing to participate. The common times standardized for the project are 6:00 am, noon, 6:00 pm and midnight. These times are random and can be selected at will, they can be at the same time daily or at a different time daily. Flexibility in programming allows groups of individuals to assume different times and time intervals to their liking. The means of synchronization will use a myriad of technological devices used mostly for other reasons including portable phones, personal computers, PDAs, music players, radios, televisions and watches as well as appliances designed specifically for the purpose. Web-site of the NNNOOOWWW project:

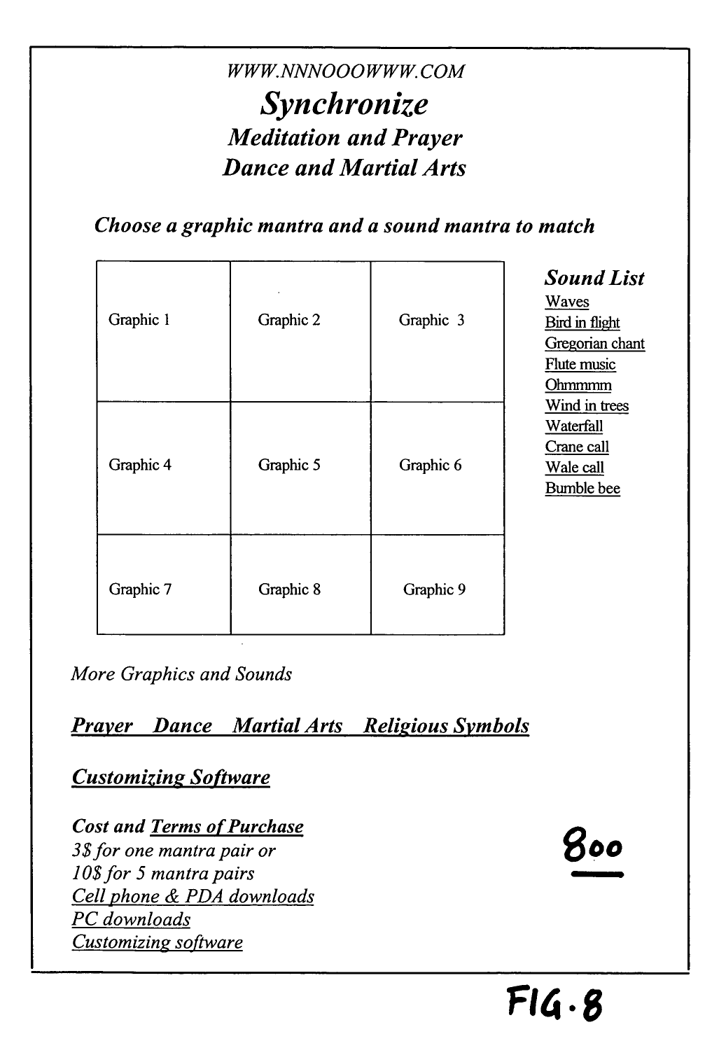

FIG. 8 is a representative diagram illustrating an embodiment of a screenshot of the homepage 800 of a prototypical website of the present invention, such as the NNNOOOWWW project.

From The Users Perspective:

A user comes to the site to purchase sound and visual mantras. From lists of different, available mantras (visual and sound combinations), one or more mantras may be purchased at a time. Sound or audio and visual mantras may be purchased for use on cell phones, other portable phones, PCs or PDAs.

On the web-site, the user is able to experience all the mantras for phones, PDAs and PCs. The sizes of the visual mantras are adjustable to the sizes of screens or monitors on cell phones, PDAs and several standard PC monitor sizes (e.g., 1/16, ⅛, ¼, ½ and full screen). The volume and other characteristics of the sound mantras are adjustable. Downloaded mantra software (graphics, sounds and menu) allows the user to assign certain buttons, or one or more joy sticks, for control and operation of the applet with a limited instruction set including quenching sounds and effects including fading in and out of images. Programming tools for such applets or downloadable executable programs are available for use with miniature or other personal electronics. The sound mantras may be instructed to sound once, twice, three times or more times with or without visual mantras. Their durations may also be modified or adjustable. Graphic mantras may occur without sound.

The user of the site may also purchase software that allows him/herself to customize a mantra, such as by using his/her own graphics and sound files, and also implementing or facilitating meditation or contemplation at non-traditional times if desired, i.e., “traditional” times being at 6 am, noon, 6 pm and midnight.

The web-site is active 24/7. In one embodiment, at 6 am, noon, 6 pm and midnight sales are suspended to honor a minute of contemplation. The mantra of “peace” will repeat in many languages over the minute. The religious-oriented or non-denominational mantra will be a graphic such as the opening of a lotus blossom with time lapsed photography over 60 seconds. The web-site's business transactions are suspended at these times.

A ringtone is the melody that is played on a mobile handset, when the handset is receiving a call. Typically one can change melody or ringtone tone as many times as one likes and make the handset sound as original as possible. A picture message is a graphic that can be used with a short message or used to replace a short message. A picture message is another creative way of communicating.

A user typically browses through the site for their choice of mantra, notes the ID number of the specific mantra or meditation or contemplation minder to download, dial the 900 or other toll/non-toll telephone number and follow the prompts. One is asked to input the phone number of the mobile phone to which to send the item. One may also be asked to input the ID number of the item desired to be downloaded. On completion of the call, the mantra or other meditation minder files will be sent to the electronic device, such as a mobile phone. This will typically take about a minute or more or less. When the mantra or meditation minder files arrive, the users cell phone or other electronic device to which the files were downloaded will alert the user with a message indicating ‘received’. The user can play and review the sound and graphic files.

It will be clear that there will be an infinite number of ways of designing and implementing the various menus and control functions of the downloadable meditation minder files. In one embodiment, the user selects ‘options’. The user will then be offered the choice of ‘install’, ‘show’, ‘setup’, ‘playback’, ‘save’ ‘modify’, ‘forward’, ‘discard’, or other. It will be understood that different models of cell phones, PDAs, etc., will have varying capacities for storing and installing such downloadable mantras or meditation minder files, and will also utilize different and varying operating standards and systems, depending on manufacturer and model, etc.

Description of Visual and Sound Mantras:

They are queues to meditation, contemplation, prayer, or physical movement including dance. They span the length of 60 seconds, or more or less, and can start at 6:00 am, noon, 6:00 pm and midnight or at other times.

Visual mantras are single or multiple frame graphics, such as a video sequence, and may be ring tones, logos, picture messages, poly tones, color images, Java games, True Tones, MP3s, color animations, other color images and video clips, photographs or art work or animation. JPG and JPEG, MPG and MPEG, VOC, MOV, WAV, Windows Media Player, Real Player, HTML, other Java files, other types of files may be utilized to communicate the downloadable mantras consisting of data and instruction files, in any of standard or proprietary or custom operating language, software application or other executable file. The mantras, once installed on the platform of choice, such as a cell phone, PDA, game or music module or console, or other portable or stationary electronic device, may fade in and out, one or many times over the period of 60 seconds. The visual mantras are accompanied by sound mantras. The sound mantras may fade in and out as the visual mantras may also.

The sound mantras may be quenched by pressing any key on the device or using a joy stick if the device has one. To un-quench the quenched mantra the “S” key may be pressed or the joy stick may be used.

The visual mantras may be quenched by pressing any key on the device or using a joy stick if the device has one. To un-quench the quenched mantra the “M” or other key or keystroke combination may be pressed or the joy stick may be used.

In an embodiment, if the mantra occurs during a phone conversation, it may be held in limbo or in waiting, such as in a holding state, for activation automatically or by pressing the “A” or other key at the time the user feels appropriate, or it may be shared with the individual or individuals sharing the phone conversation in real time or subsequently. In another embodiment, if the mantra is received while the telephone or other portable or stationary electronic device is being used, it is simply played simultaneously, i.e, over the same or a separate channel or through the same or different speakers, optionally in a duplex or VoIP protocol or format.

In one embodiment of the present invention, mantras sold at the web-site may represent the brand of a product or be a corporate logo or a representation of a corporation.

In one embodiment of the present invention, the agreement signed on purchase will acknowledge that each mantra is for personal use and not for distribution. In one embodiment, arrangement for group purchasing of software will be facilitated at the web-site or elsewhere, and in another embodiment a producer of cell phones or other devices may install the mantras directly on their devices.

The layout and workings of the web-sites may change, as may be desired. Several or many similar web-sites selling mantras may exist. This will in part be determined by the licensing arrangements for regions of the globe or countries to be discussed later.

The WWW.NNNOOOWWW.com Web-Site:

In one embodiment of the present invention, the visual mantras are presented as 9 (in a three by three array) mantras per page. The first page of the web-site will not have any mantras with religious connotations, but could in other embodiments. The lotus blossom is generally considered without religious connotations and a lotus blossom mantra will be the center of the nine mantra array. Each frame (member of the array) may be clicked on to view and hear the mantra. A sound mantra is attached to each visual mantra, but a buyer may opt to choose another sound mantra with a given visual mantra.

Programming of the web-site will make the combination of visual and sound mantras as easy as possible. It may be necessary to have a search function of both visual and sound mantras if the licensed developer of web-site/sales platform, mantras and mantra software markets a large amount of mantras.

Meditation Software Requirements:

The products sold on the www.nnnooowww.com include:

Files of graphic images:
Single graphic images
Sequences of graphic images
Patterns and graphics generated from mathematic formulation
Animations

Files of sound:
Repeated sounds
Sequences of sounds
Music
Sounds or music generated from mathematic formulation

Software:
Used to control the timing and duration of queues
   Standard queue times are 6:00 am, noon, 6:00 pm and midnight PCT
   Standard queue duration is one minute
Used to control the quenching of sound and visual queues on appliances
Functioning to assure that graphic and sound files are downloaded to a particular appliance or a particular phone number

Customizing Software:
Designed to facilitate the construction of unique graphic and sound queues by individuals
Designed to facilitate unique timing in use of queues

Each time software products including graphic and sound files and software are downloaded from www.nnnooowww.com site to an electronic appliance, like a cellular phone, PDA or PC, the device's unique identification number will be used to assure that the software products may not be transferred to other appliances for which they were not purchased. If the software products are purchased for a cellular phone number, the software products may be transferrable to another phone using the same number. The term “queues” shall refer to queues for meditation, contemplation, prayer and physical movement including dance and martial arts.

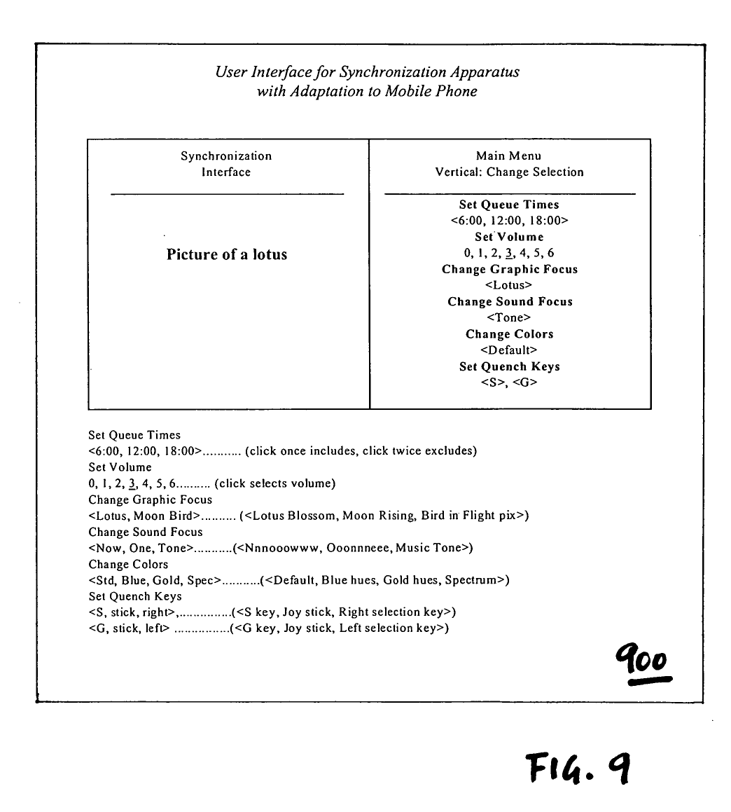

FIG. 9 is a representative diagram illustrating a preferred embodiment of a screenshot of a web page or user interface for a synchronization apparatus adapted for use with a cellular or other mobile phone 900. In a preferred embodiment, the “Graphic Focus” menu item, when selected presents either: A short list of possible graphics files to select from or a scrolling list of graphic files. In a preferred embodiment, the “Sound Focus” menu item, when selected presents either a short list of possible sound files to select from or a scrolling list of sound files.

### Conclusion

The present invention is the method and apparatus, using components of electronic and/or mechanical devices contained in part within or completely within watches, personal desk accessories, portable phones, computers, networked devices or appliances, to temporally synchronize meditation and/or contemplation including prayer and/or physical movement in individuals, in shared or diverse physical surroundings. The form of the cues, which occur one or several times a day for a group of individuals, are in sound and visual cues including images, color patterns and animation.

The potential spiritual and psychological benefits of the present invention include enhanced harmony of spiritualism, reinforcement of the constructive affects of faith, and/or of a sense of belonging to a group and/or a sense of belonging to a higher power. The promotion of responsible materialism within society is a potential outcome if the synchronization and reenforcement of the aforementioned activities contributes to the therapeutic remedy for narcissism, borderline personality and anxiety disorders.

The process of using watches and other devices to synchronize meditation has ramifications to those individuals wishing to synchronize their meditation with others. Without this process the use of and adherence to explicit instructions is needed to facilitate the uses of watches for this purpose.

Psychological descriptions of personality frequently define patterns of the mechanisms individuals use to defend against pain, fear and a diminished sense of self. Equivalently significant to defining personality are mechanisms used constructively to strengthen psychological factors which balance pain and fear and which facilitate acceptance and motivate healthy assertiveness. Included in these mechanisms are the reinforcement of faith, a sense of belonging and a sense of security. The present invention may result in the strengthening of an individual's sense of and commitment to faith and sense of belonging when meditation, contemplation (including prayer), dance, exercise and the practice of martial arts are synchronized and practiced as a group. As an adjuvant to said activities the present invention promotes their use.

The positive affects of faith and sense of belonging, when reinforced, may diminish the need of individuals to use psychological defense mechanisms of control, paranoia, compulsive processes and the transference of anger.

An additional ramification of the present invention is that it may bring happiness to those individuals wishing to be involved in said activities in synchrony with other individuals.

Unless defined otherwise, all technical and scientific terms used herein have the same meaning as commonly understood by one of ordinary skill in the art to which the present invention belongs. Although any methods and materials similar or equivalent to those described can be used in the practice or testing of the present invention, preferred methods and materials are now described. All publications and patent documents referenced in the present invention are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

1. A method for temporal synchronization for a plurality of individuals, the method comprising the following steps:
Selecting a preferred meditation minder from a group of meditation minders consisting of at least one downloadable audio and visual file located on a remote server;
Downloading the preferred meditation minder to a local electronic device; and
Installing and setting up the meditation minder on the local electronic device; and
Providing an audible and visual cue from the local electronic device to engage in a contemplative activity for a predetermined time interval at the desired, pre-selected time points throughout every day:

2. The method of claim 1 in which the at least one downloadable audio and visual file comprises one or more of the group of file types consisting of the following: single or multiple frame graphics, video sequence, ringtones, logos, picture messages, poly tones, color images, Java games, True Tones, MP3s, color animations, other color images and video clips, photographs or art work or animation. JPG and JPEG, MPG and MPEG, VOC, MOV, WAV, TXT, DOC, WPD, PDF, Windows Media Player, Real Player, HTML, and other Java files.

3. The method of claim 1 wherein the local electronic device comprises one or more of the electronic devices within the group consisting of wristwatches, clocks, 2-way communication devices, cellular and mobile telephones, networked computer devices including computers, home entertainment processors, televisions and radios, information management tools, PDAs, and a dedicated temporal synchronization cuing appliance.

4. A contemplative and meditative information distribution system comprising: an information broadcasting terminal operated by a particular source of contemplative and meditative information for downloadable broadcasting cues for engaging in contemplative and meditative activities; means for receiving the downloadable broadcast cues; and means for display and broadcast of the cue to a user.

5. A method for treatment of a group of patients each having psychological and psycho-social disorders, the method for treatment based on cued repetitive practice of prescribed activities, the method comprising the following steps:
establishing a network for downloading contemplative activity cueing signals to a plurality of remote synchronization devices; and
providing a group of patients each with a temporal synchronization device;
downloading one or more of the contemplative activity cueing signals to the plurality of remote synchronization devices, thereby inducing temporal synchronization of the cued repetitive practice of prescribed activities within the group of patients.

6. A system for temporal synchronization of a contemplative activity by a plurality of individuals, the system comprising:
An electronic computer network;
A server computer coupled to the electronic computer network, the server computer containing one or more downloadable meditation minder files and a user interface; and
A local electronic device in the group of electronic devices consisting of: wristwatches, clocks, 2-way communication devices, cellular and mobile telephones, networked computer devices including computers, home entertainment processors, televisions and radios, information management tools, PDAs, and a dedicated temporal synchronization cuing appliance, wherein the one or more downloadable meditation minder files can be accessed through the user interface located on the server computer and downloaded to the local electronic device to provide cues to engage in contemplative activity one or more times per day, as desired.
